(19) United States
(12) Patent Application Publication
(10) Pub. No.: US 2011/0306773 A1
(43) Pub. Date: Dec. 15, 2011

(54) PROCESS FOR PREPARATION OF METAXALONE

(76) Inventor: Battula Srinivasa Reddy, Ameerpet (IN)

(21) Appl. No.: 13/084,848

(22) Filed: Apr. 12, 2011

(30) Foreign Application Priority Data
Apr. 12, 2010 (IN) 1014/CHE/2010

(51) Int. Cl. C07D 263/06

(52) U.S. Cl. 548/232

(57) ABSTRACT

An improved process for the preparation of Metaxalone of formula I

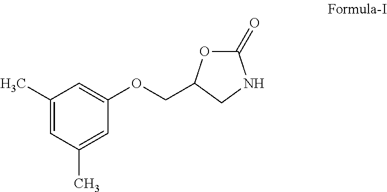

wherein, reacting 3,5-dimethyl phenol with 3-chloro-1,2-propane diol in presence of a base, to produce 3-(3,5-dimethylphenoxy)propane-1,2-diol which is further reacted with urea in presence of polyalkylene glycol to obtain Metaxalone, which is optionally crystallized in ethyl acetate in presence of mineral acid.

### FIELD OF THE INVENTION:

The present invention relates to an improved process for the preparation of Metaxalone of Formula I, which is optionally converted to the pharmaceutically acceptable salts thereof.

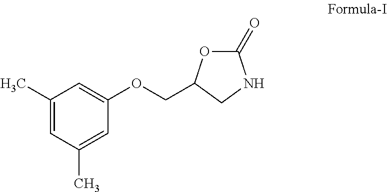

### BACKGROUND OF THE INVENTION:

Metaxalone is, chemically known as 5-[(3,5-dimethylphenoxy)methyl]-2-oxazolidinone, which is represented by a structural formula I

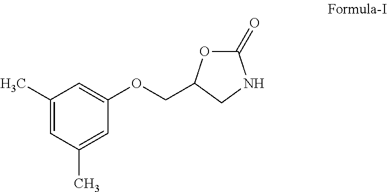

Metaxalone is a skeletal muscle relaxant, marketed under the trade name SKELAXIN®. Skelaxin is indicated as an adjunct to rest, physical therapy and other measures for the relief of discomforts associated with acute, painful musculoskeletal conditions. The mode of action of this drug has not been clearly identified but may be related to its sedative properties. Metaxalone does not directly relax tense skeletal muscles in man. The commercially available tablet contains Metaxalone, 400 mg along with inert compression tableting excipients.

Metaxalone was first disclosed in U.S. Pat. No. 3,062,827, which is assigned to A. H. Robins Company Inc. This patent discloses different routes of synthesis for the preparation of Metaxalone. One such process is by reacting 3-(3′,5′-dimethylphenoxy)-1,2-propanediol and urea at a temperature of 195-200° C. Alternatively, the selected 3-phenoxy-1-chloro-2-propanol may be reacted with urea instead of the phenoxy-1,2-propanediol under the same conditions. The 5-(3′,5′-dialkylphenoxymethyl)-2-oxazolidones may also be prepared by reacting a selected 3-phenoxy-2-hydroxy-l-propylcarbamate and urea in equimolar quantities and at elevated temperatures. However, the process disclose in this patent has some problems, like the low purity levels of the final compound.

U.S. Pat. No. 3,446,814 assigned to Henkel & Cie GmbH discloses a process for the preparation of Metaxalone by reacting triglycidyl isocyanurate with m-xylenol. The patent exemplifies reacting the raw materials with pulverized sodium hydroxide in chlorobenzene at 131-132° C. for 13 hrs in presence of benzyltrimethylammonium chloride, followed by recrystallization of the product from chlorobenzene. This patent does not disclose the purity of Metaxalone, further this process is an energy consuming process.

GB 1104773 assigned to Henkel & Cie GmbH discloses the process for the preparation of Metaxalone by reacting the 3,5-dimethyl-phenol with triglycidyl isocyanurate in an organic solvent using a phenol/isocyanurate molar ratio of about 1:3. The present invention is directed to a more efficient and economic synthesis of Metaxalone.

U.S. Pat. No. 6,538,142, assigned to Farchemia S.r.l. discloses a process for the preparation of Metaxalone by reacting 3,5-dimethylphenol with epichlorohydrin to obtain a mixture of 1-(3,5-dimethylphenoxy)2,3-epoxy propane and 1-(3,5-dimethylphenoxy)-3-chloro-2-propanol, which is reacted with benzylamine and on further reduction and reaction with dimethylcarbonate in the presence of a strong base to produce 5-(3,5-dimethylphenoxymethyl)-2-oxazolidinone.

WO 2008/006096 assigned to Boehringer Ingelheim Chemicals, Inc discloses a process comprising reacting 3-(3,5-dimethylphenoxy)-1-amino-2-propanol with methyl carbamate in the presence of a strong base to obtain Metaxalone.

The prior art processes disclosed involves either many number of reaction steps or usage of costly reagents like Pd/C.

The present inventors have now found a much simpler, cost effective process when compared to other prior art process.

### OBJECTIVE OF THE INVENTION:

The main object of the present invention is to provide an improved, simple, cost effective and industrially advantageous process for the preparation of Metaxalone with high purity and high yields on commercial scale.

### SUMMARY OF THE INVENTION:

The main objective of the present invention is to provide an improved process for the preparation of Metaxalone of formula I

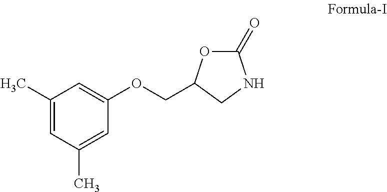

wherein, reacting 3,5-dimethyl phenol with 3-chloro-1,2-propane diol in presence of a base, to produce 3-(3,5-dimethylphenoxy)propane-1,2-diol which is further reacted with urea in presence of polyalkylene glycol to obtain Metaxalone, which is optionally crystallized in ethyl acetate in presence of mineral acid.

### DETAILED DESCRIPTION OF THE INVENTION:

The present invention relates to an improved process for the preparation of Metaxalone by reacting 3,5-dimethyl phenol with 3-chloro-1,2-propane diol in presence of a base, at a temperature of about 80-100° C. The product 3-(3,5-dimethylphenoxy) propane-1,2-diol is filtered using an organic solvent. To the poly(alkyleneglycol), 3-(3,5-dimethylphenoxy) propane-1,2-diol is added at room temperature and the reaction mixture heated to 60-90° C. and slowly raise the temperature to about 150-160° C. To this reaction mixture molten urea is slowly added at a temperature of about 150-160° C. and heated to 200-205° C. to produce Metaxalone. The crude Metaxalone is crystallized using Ethyl acetate in presence of small quantities of mineral acid.

The process is schematically represented as shown in scheme I:

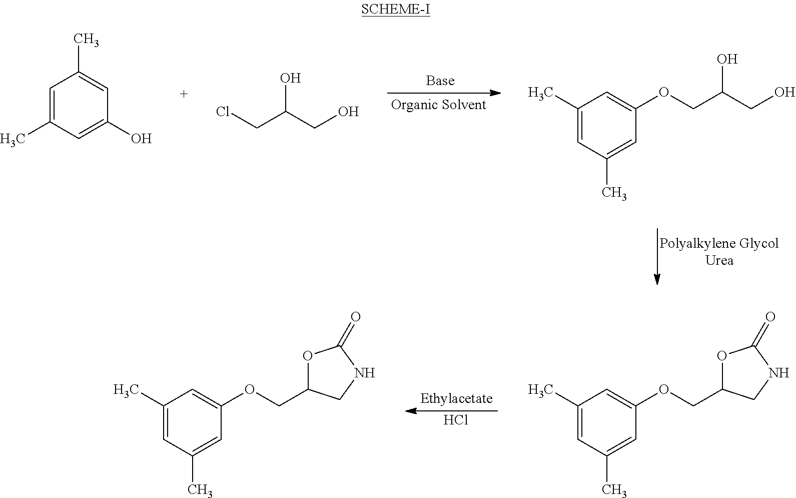

According to one embodiment of the present invention base is selected from the group comprising of sodium hydroxide, potassium hydroxide, sodium carbonate, sodium bicarbonate, potassium carbonate and potassium bicarbonate.

According to another embodiment of the present invention organic solvent is selected from the group comprising of toluene, xylene, cyclohexane, monochlorobenzene, methylene chloride, ethylene chloride or mixtures thereof.

According to another embodiment of the present invention poly(alkyleneglycol) is selected from the group comprising of poly(ethyleneglycol) (PEG) with an average molecular weight in the range between 200 to 10,000, preferably PEG-400. The poly(alkyleneglycol) is used as reaction facilitator.

According to one more embodiment of the present invention mineral acid is selected from Hydrochloric acid, Hydrobromic acid.

While the present invention has been described in terms of its specific embodiments, certain modifications and equivalents will be apparent to those skilled in the art and are intended to be included within the scope of the present invention.

The present invention can be illustrated in one of its embodiment by the following non-limiting examples.

### EXAMPLES

### Example 1: Preparation of Metaxalone

3 gm of sodium hydroxide in DM water was added to 2 gm of 3,5-dimethyl phenol at room temperature room temperature and external cooling was applied to reaction mixture. To this reaction mass 8 gm of 3-chloro-1,2-propane diol was added and the temperature was slowly raised to about 100° C. To the crude product obtained 3-(3,5-dimethylphenoxy)propane-1,2-diol, 30 ml toluene was added and to the obtained pure 3-(3,5-dimethylphenoxy)propane-1,2-diol 30 ml of polyethylene glycol was added at room temperature and the reaction mixture was heated to 80° C. and slowly raised to 160° C. To this reaction mixture 10 gm molten urea was slowly added and heated to 205° C. to produce Metaxalone. 100 ml of Ethyl acetate was added to the crude Metaxalone obtained and treated with 2 ml of HCl. The product was filtered and dried to obtain Metaxalone. Purity—99.5%; Yield —95%.

### Example 2: Preparation of Metaxalone

6 gm of sodium hydroxide in DM water was added to 4 gm of 3,5-dimethyl phenol at room temperature room temperature and external cooling was applied to reaction mixture. To this reaction mass 8 gm of 3-chloro-1,2-propane diol was added and the temperature was slowly raised to about 80° C. To the crude product obtained 3-(3,5-dimethylphenoxy)propane-1,2-diol, 60 ml toluene was added and to the obtained pure 3-(3,5-dimethylphenoxy)propane-1,2-diol 60 ml of polyethylene glycol was added at room temperature and the reaction mixture was heated to 60° C. and slowly raised to 160° C. To this reaction mixture 20 gm molten urea was slowly added and heated to 200° C. to produce Metaxalone. 200 ml of Ethyl acetate was added to the crude Metaxalone obtained and treated with 4 ml of HCl. The product was filtered and dried to obtain Metaxalone. Purity—99%; Yield —95%.

What is claimed is:

1. A method of preparing Metaxalone, comprising the steps of:
reacting 3,5-dimethyl phenol with 3-chloro-1,2-propane diol in presence of a base;
isolating 3-(3,5-dimethylphenoxy)propane-1,2-diol; and
reacting 3-(3,5-dimethylphenoxy)propane-1,2-diol with urea in presence of polyalkylene glycol to obtain Metaxalone.

2. The method according to claim 1, further comprising the step of crystallizing in ethyl acetate in presence of mineral acid.

3. The method according to claim 1, wherein the base is selected from the group comprising of sodium hydroxide, potassium hydroxide, sodium carbonate, sodium bicarbonate, potassium carbonate and potassium bicarbonate.

4. The method according to claim 1, wherein the organic solvent is selected from the group comprising of toluene, xylene, cyclohexane, monochlorobenzene, methylene chloride, ethylene chloride or mixtures thereof.

5. The method according to claim 1, wherein the poly(alkyleneglycol) is selected from the group comprising of poly(ethyleneglycol) (PEG) with an average molecular weight in the range between 200 to 10,000, preferably PEG-400.

6. The method according to claim 1, wherein the mineral acid is selected from Hydrochloric acid, Hydrobromic acid.
